(19) United States
(12) Patent Application Publication
(10) Pub. No.: US 2026/0325705 A1
(43) Pub. Date: Oct. 22, 2026

(54) METHOD FOR PREPARING A HAEMOSTATIC COMPOSITION

(71) Applicants: Ferrosan Medical Devices A/S, Søborg (DK); Ethicon Inc., Raritan, NJ (US)

(72) Inventors: Peter Lund Hammershøj, Brønshøj (DK); Kristian Larsen, Værløse (DK); Douglas B. Johns, Milford, NJ (US); Nicole Smith, Hoboken, NJ (US); Michael Cardinale, Morristown, NJ (US); Gabriella Ferrara, Old Tappan, NJ (US); Guanghui Zhang, Belle Mead, NJ (US)

(21) Appl. No.: 19/444,637

(22) Filed: Jan. 9, 2026

Related U.S. Application Data
(63) Continuation of application No. 18/470,768, filed on Sep. 20, 2023, now Pat. No. 12,544,483.
Application No. 18/470,768 is a continuation of application No. 16/407,503, filed on May 9, 2019, now Pat. No. 11,801,324.
(60) Provisional application No. 62/669,056, filed on May 9, 2018.

(51) Int. Cl. A61L 24/04; A61B 17/00; A61B 17/12; A61L 24/00

(52) U.S. Cl. CPC A61L 24/043; A61B 17/12186; A61L 24/0042; A61B 2017/00495; A61L 2400/04; A61L 2400/06

(57) ABSTRACT

The present disclosure relates to a method for preparing a haemostatic composition comprising thrombin, the method comprising the step of reconstituting a dry thrombin directly in a paste, such as a paste comprising a biocompatible polymer. The haemostatic composition comprising thrombin may be prepared from a dry thrombin composition and a paste in a single step operation and be used for treatment of a wound.

### RELATED APPLICATIONS

This application is a continuation of U.S. application Ser. No. 18/470,768, filed Sep. 20, 2023, which is a continuation of U.S. application Ser. No. 16/407,503, filed on May 9, 2019, now U.S. Pat. No. 11,801,324, issued on Oct. 31, 2023, which claims the benefit of U.S. Provisional Application No. 62/669,056, filed on May 9, 2018. The entire teachings of the above applications are incorporated herein by reference.

### TECHNICAL FIELD

The present disclosure relates to a simplified method for preparing a haemostatic paste composition comprising thrombin, the method comprising the step of reconstituting a dry thrombin directly in a paste, such as a paste comprising a biocompatible polymer. The haemostatic composition comprising thrombin may be prepared from a dry thrombin composition and a paste in a single step operation and be used for treatment of a wound.

### BACKGROUND

Protein-based haemostatic materials such as collagen and gelatine are commercially available in solid sponge and loose or unpacked powder form for use in surgical procedures. Mixing of the loose or unpacked powder with a fluid such as saline or a thrombin solution may form a paste or slurry that is useful as a haemostatic composition for use in cases of diffuse bleeding, particularly from uneven surfaces or hard to reach areas, depending on mixing conditions and relative ratios of the materials.

Conventional haemostatic pastes are usually prepared at the point of use by mechanical agitation and mixing of a biocompatible polymer, e.g. gelatine, and a liquid, e.g. a thrombin solution, to provide uniformity of the composition. Mixing to form a paste usually requires extensive mixing, such as kneading or transfer between two syringes.

It is often desired that the haemostatic paste comprises a thrombin component to provide optimal haemostatic effect of the paste. Due to stability reasons, the thrombin component is usually provided as a dry composition separate from the biocompatible polymer component. The dry thrombin is then reconstituted to form a suspension or solution before mixing with the biocompatible polymer. This step of reconstitution of the thrombin component usually takes place immediately prior to mixing with the biocompatible polymer. Reconstitution of thrombin is time consuming and challenging with multi-step syringe handlings involved; factors which are undesirable in an operating room setting with bleedings, as the surgeon will have to abrupt his procedure while waiting for the haemostat to be prepared.

Surgiflo® Haemostatic Matrix (Ethicon) is a kit for producing a haemostatic gelatine paste comprising thrombin, which is prepared by first reconstituting a dry thrombin composition and subsequent transferring a gelatine matrix-thrombin solution mixture back and forth between two connected syringes for a total of at least six passes.

Floseal® Haemostatic Matrix (Baxter) is likewise a kit for producing a haemostatic gelatine paste, requiring initial reconstitution of a dry thrombin composition followed by transfer of the gelatine matrix-thrombin solution mixture back and forth between two connected syringes for a total of at least twenty passes. Once a substantially homogenous paste composition is achieved, the haemostatic pastes can be applied to a bleeding to promote haemostasis by extruding the pastes from the syringe.

Attempts have also been made to provide the biocompatible polymer and the thrombin in dry form in the same syringe, such as described previously in e.g. WO 2011/151400, WO 2011/151384, WO 2011/151386 and WO 2013/185776, the teachings of which are incorporated by reference in their entirety. However, due to the sensitivity of thrombin to the sterilisation methods usually employed in the manufacture of haemostatic products, i.e. ionising radiation and/or ethylene oxide, the sensitivity of thrombin to water and the different physical-chemical properties of thrombin and the polymers usually employed, such as gelatine, it has proven challenging to manufacture such “all-in-one” products so that they retain sufficient thrombin activity during manufacturing, sterilisation and throughout the shelf-life of the product and/or which ensures satisfactory distribution of thrombin in the final reconstituted haemostatic paste product.

As mentioned above, incorporation of thrombin in the haemostatic paste is challenging due to either manufacturing or stability reasons or due to time consuming reconstitution of a dry thrombin composition prior to mixing with the biocompatible polymer. Thus, there is a need in the art for developing novel methods for quick and easy incorporation of thrombin in the haemostatic paste.

### SUMMARY

The present disclosure addresses the above problems relating to incorporation of thrombin in haemostatic pastes and provides a method for reconstituting a dry thrombin composition directly in a paste comprising a biocompatible polymer, to generate a haemostatic paste comprising thrombin in a single step operation. Such simple and fast method for preparing a haemostatic composition is highly valuable in the operating room where potential bleeding must be controlled in a fast and efficient manner.

Thus, in one aspect, the present disclosure relates to a method for preparing a haemostatic composition, the method comprising the steps of:
a) providing a dry thrombin composition in a first container;
b) providing a paste comprising a biocompatible polymer in a second container;
c) connecting the first container and the second container using suitable connecting means; and
d) mixing the contents of the containers.

In a second aspect, the present disclosure relates to a method for reconstituting a dry thrombin composition, the method comprising the steps of:
a) providing a dry thrombin composition in a first container;
b) providing a paste comprising a biocompatible polymer in a second container;
c) connecting the first container and the second container using suitable connecting means; and
d) mixing the contents of the containers.

The mixing of the contents of the containers may be performed by transferring the contents of the containers back and forth a number of times, such as less than 20 times, for example less than 15 times, such as less than 10 times, preferably about six times.

The inventors have surprisingly found that such method for reconstituting a dry thrombin composition directly in a paste comprising a biocompatible polymer results in a substantially homogenous distribution of the thrombin in the haemostatic composition. Thus, the present disclosure provides a method for preparing a haemostatic composition comprising thrombin in a fast and simple manner which does not require separate reconstitution of the thrombin component prior to mixing with the biocompatible polymer.

The present disclosure further relates to a haemostatic composition obtainable by the methods as described herein as well as to uses thereof.

### DESCRIPTION OF DRAWINGS

FIG. 1

Displays an exemplary setup and method for reconstituting a dry thrombin composition directly in a paste comprising a biocompatible polymer to generate a haemostatic composition. The dry thrombin composition is contained in a first syringe and the paste comprising a biocompatible polymer, here exemplified by a gelatine paste, is contained in a second syringe. The two syringes are interconnected, in this embodiment via a static mixer, and the content of the syringes is transferred back and forth six times to generate the haemostatic composition.

FIG. 2

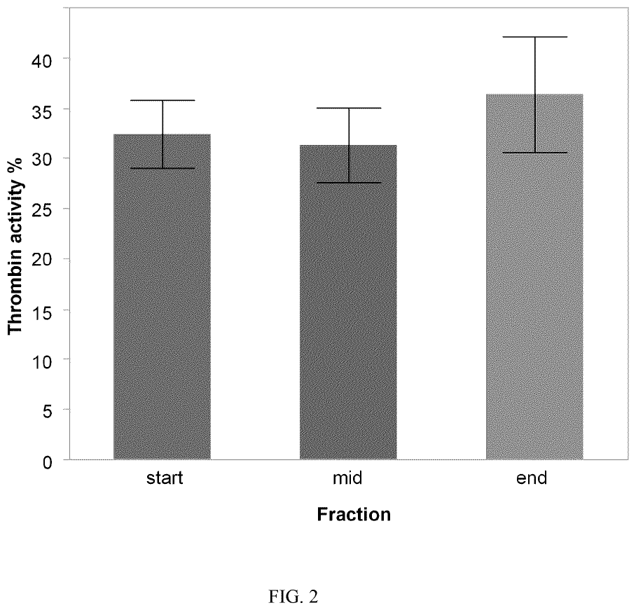

Displays thrombin activity distribution in the start, mid and end portion of a syringe comprising a haemostatic composition reconstituted using the method of the present disclosure. The mean thrombin activity is given as percentage of the total thrombin activity. The data shows that reconstitution of dry thrombin with a paste yields a haemostatic paste composition having a homogeneous distribution of thrombin. Error bars are constructed using one standard deviation from the mean.

FIG. 3

Displays haemostatic efficacy (Time to Haemostasis (TTH)) of a paste of the present invention (6TM paste) compared to control (Surgiflo mixed with 2 ml thrombin solution) (Mean+/−SEM, n=7). The 6TM paste prepared according to the present invention was found to induce haemostasis faster and more consistently than the control paste.

### Definitions

A “bioactive agent” is any agent, drug, compound, composition of matter or mixture which provides some pharmacologic, often beneficial, effect that can be demonstrated in vivo or in vitro. An agent is thus considered bioactive if it has interaction with or effect on a cell tissue in the human or animal body. As used herein, this term further includes any physiologically or pharmacologically active substance that produces a localized or systemic effect in an individual. Bioactive agents may be a protein, such as an enzyme. Further examples of bioactive agents include, but are not limited to, agents comprising or consisting of an oligosaccharide, a polysaccharide, an optionally glycosylated peptide, an optionally glycosylated polypeptide, an oligonucleotide, a polynucleotide, a lipid, a fatty acid, a fatty acid ester and secondary metabolites. It may be used either prophylactically, therapeutically, in connection with treatment of an individual, such as a human or any other animal. The term “bioactive agent” as used herein does not encompass cells, such as eukaryotic or prokaryotic cells.

“Biocompatible” refers to a material's ability to perform its intended function without eliciting any substantial undesirable local or systemic effects in the host.

“Biologically absorbable” or “resorbable” are terms which in the present context are used to describe that the materials of which the said powder are made can be degraded in the body to smaller molecules having a size which allows them to be transported into the blood stream. By said degradation and absorption the said powder materials will gradually be removed from the site of application. For example, gelatine can be degraded by proteolytic tissue enzymes to absorbable smaller molecules, whereby the gelatine, when applied in tissues, typically is absorbed within about 4-6 weeks and when applied on bleeding surfaces and mucous membranes typically within 3-5 days.

A “gel” is a solid, jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinks within the fluid that give a gel its structure (hardness) and contribute to stickiness (tack). In this way gels are a dispersion of molecules of a liquid within a solid in which the solid is the continuous phase and the liquid is the discontinuous phase. A gel is not a paste or slurry. For example, non-crosslinked gelatine is soluble and forms a gel upon contact with an aqueous medium such as water.

“Haemostasis” is a process which causes bleeding to diminish or stop. Haemostasis occurs when blood is present outside of the body or blood vessels and is the instinctive response for the body to stop bleeding and loss of blood. During haemostasis three steps occur in a rapid sequence. Vascular spasm is the first response as the blood vessels constrict to allow less blood to be lost. In the second step, platelet plug formation, platelets stick together to form a temporary seal to cover the break in the vessel wall. The third and last step is called coagulation or blood clotting. Coagulation reinforces the platelet plug with fibrin threads that act as a “molecular glue”. Accordingly, a haemostatic compound is capable of stimulating haemostasis.

“International Unit (IU)”. In pharmacology, the International Unit is a unit of measurement for the amount of a substance, based on biological activity or effect. It is abbreviated as IU, UI, or as IE. It is used to quantify vitamins, hormones, some medications, vaccines, blood products, and similar biologically active substances.

A “paste” according to the present disclosure has a malleable, putty-like consistency, such as toothpaste. A paste is a thick fluid mixture of pulverized solid/solid in powder form with a liquid. A paste is a substance that behaves as a solid until a sufficiently large load or stress is applied, at which point it flows like a fluid, i.e. a paste is flowable. Flowables conform efficiently to irregular surfaces upon application. Pastes typically consist of a suspension of granular material in a background fluid. The individual grains are jammed together like sand on a beach, forming a disordered, glassy or amorphous structure, and giving pastes their solid-like character. It is this “jamming together” that gives pastes some of their most unusual properties; this causes a paste to demonstrate properties of fragile matter. A paste is not a gel/jelly. A “slurry” is a fluid mixture of a powdered/pulverized solid with a liquid, such as water. Slurries behave in some ways like thick fluids, flowing under gravity and being capable of being pumped if not too thick. A slurry may functionally be regarded as a thin, watery paste, but a slurry generally contains more water than a paste. Substantially water-insoluble powder particles, such as cross-linked gelatine particles, will form a paste upon mixing with an aqueous medium.

“Percentage”. If nothing else is indicated, the percentage is percentage by weight: % w/w or wt %. Ratios are indicated as weight by weight (w/w).

“Variation in thrombin content”. The variation in thrombin content as used herein is defined as the percentage difference in the mean thrombin activity between two fractions of haemostatic composition. The mean thrombin activity is given as thrombin activity of a fraction as a percentage of the total thrombin activity of the full haemostatic composition.

### DETAILED DESCRIPTION

The present disclosure relates to a simplified method for preparing a haemostatic paste composition comprising thrombin. The method comprises the step of reconstituting a dry thrombin composition directly in a paste, such as a paste comprising a biocompatible polymer.

The invention thus relates to a method of preparing a haemostatic composition, the method comprising the steps of:
a) providing a dry thrombin composition in a first container;
b) providing a paste comprising a biocompatible polymer in a second container;
c) connecting the first container and the second container using suitable connecting means; and
d) mixing the contents of the containers.

After mixing, the first or the second container (depending on the number of transfers) may be used as a delivery device to deliver the haemostatic composition to tissue.

Thus, the haemostatic composition comprising thrombin may be prepared from a dry thrombin composition and a paste in a single step operation with no need for prior time-consuming and error prone reconstitution of the dry thrombin composition in a solution.

Such simple and fast method for preparing a haemostatic composition is highly valuable in the operating room where potential bleeding must be controlled in a fast and efficient manner.

The advantages of the methods provided in the present disclosure and the haemostatic composition obtained by such methods are numerous and include:
Less time spent preparing the haemostatic composition, e.g. bleeding can be stopped faster.
Decreased risk of compromising the sterility of the haemostatic composition during preparation due to less handling steps.
Decreased risk of making mistakes during preparation due to the simplified preparation of the paste.
Reliable and consistent reconstitution within a short time period.
Avoids the time-consuming and error-prone thrombin dilution steps of standard haemostatic composition preparations.
Minimises Operation Room costs since preparation of the currently described product is so simple and fast that there is no reason to pre-prepare haemostatic flowables before surgery which may not be used.
Increased flexibility to add an aqueous medium to the paste in order to modify the consistency of the final mixed composition.

All of the above factors lead to increased patient safety.

### Dry Thrombin Composition

The present disclosure relates to a method for reconstituting a dry thrombin composition directly in a paste.

Thrombin is a “trypsin-like” serine protease protein that in humans is encoded by the F2 gene. Prothrombin (coagulation factor II) is proteolytically cleaved to form thrombin in the coagulation cascade, which ultimately results in the stemming of blood loss. Thrombin in turn acts as a serine protease that converts soluble fibrinogen into insoluble strands of fibrin, as well as catalyzing many other coagulation-related reactions. In the blood coagulation pathway, thrombin acts to convert factor XI to XIa, VIII to VIIIa, V to Va, and fibrinogen to fibrin.

In one embodiment, the thrombin is human thrombin.

In one embodiment, the thrombin is recombinant human thrombin.

In other embodiments, the origin of the thrombin is from a mammal other than human, such as bovine thrombin.

In one embodiment, thrombin is in the form of prothrombin.

The dry thrombin composition may be prepared by any methods known to the skilled person and is usually provided in sterile form. Thus, in one embodiment the dry thrombin composition is sterile.

In one embodiment, the dry thrombin composition is prepared by spray-drying or freeze-drying.

In a preferred embodiment, the dry thrombin composition is prepared by freeze-drying.

In one embodiment, the dry thrombin composition comprises less than 2% water, such as less than 1% water.

The amount of thrombin in the haemostatic composition should be sufficient to ensure effective haemostasis. In one embodiment the final concentration of thrombin in the haemostatic composition is in the range of about 50 IU/mL to about 1000 IU/mL, for example 100 IU/mL to about 500 IU/mL, such as about 150 IU/mL to about 450 IU/mL, for example about 200 IU/mL to about 400 IU/mL, such as about 200 IU/mL to about 300 IU/mL.

The dry thrombin composition may optionally comprise one or more hydrophilic agent(s), such as for example one or more polyol(s) and/or one or more poly(ethylene glycol) (s) (PEGs).

In one embodiment, the dry thrombin composition comprises one or more further bioactive agent(s). Such one or more bioactive agent(s) may be able to stimulate haemostasis, wound healing, bone healing, tissue healing and/or tendon healing.

In one embodiment, the dry thrombin composition optionally comprises one or more extrusion enhancer(s), such as for example albumin. The use of extrusion enhancers to improve extrudability of haemostatic pastes is e.g. described in WO 2015/086028, which is hereby incorporated in its entirety.

### Paste Comprising Biocompatible Polymer

The dry thrombin composition of the disclosure is reconstituted directly in a paste, such as a paste comprising a biocompatible polymer.

The biocompatible polymer of the present disclosure may be a biologic or a non-biologic polymer. Suitable biologic polymers include proteins, such as gelatine, collagen, albumin, hemoglobin, casein, fibrinogen, fibrin, fibronectin, elastin, keratin, and laminin; or derivatives or combinations thereof. Particularly preferred is the use of gelatine or collagen, more preferably gelatine. Other suitable biologic polymers include polysaccharides, such as glycosaminoglycans, starch derivatives, xylan, cellulose derivatives, hemicellulose derivatives, agarose, alginate, and chitosan; or derivatives or combinations thereof. Suitable non-biologic polymers will be selected to be degradable by either of two mechanisms, i.e. (1) break down of the polymeric backbone or (2) degradation of side chains which result in aqueous solubility. Exemplary nonbiologic polymers include synthetics, such as polyacrylates, polymethacrylates, polyacrylamides, polyvinyl resins, polylactide-glycolides, polycaprolactones, and polyoxyethylenes; or derivatives or combinations thereof. Also combinations of different kinds of polymers are possible.

In one embodiment, the biocompatible polymer is biologically absorbable. Examples of suitable biologically absorbable materials include gelatine, collagen, chitin, chitosan, alginate, cellulose, oxidised cellulose, polyglycolic acid, polyacetic acid and combinations thereof. It will be understood that various forms thereof, such as linear or cross-linked forms, salts, esters and the like are also contemplated for the present disclosure. In a preferred embodiment of the invention, the biologically absorbable material is gelatine. Gelatine is preferred since gelatine is highly biologically absorbable. Furthermore, gelatine is highly biocompatible, meaning that it is non-toxic to an animal, such as a human being, when/if entering the blood stream or being in long-term contact with human tissues.

The gelatine typically originates from a porcine source, but may originate from other animal sources, such as from bovine or fish sources. The gelatine may also be synthetically made, i.e. made by recombinant means.

In a preferred embodiment the biocompatible polymer is cross-linked. Cross-linking usually renders the polymer substantially insoluble in an aqueous medium. In one embodiment, the biocompatible polymer consists of powder particles which are substantially insoluble in an aqueous medium. Any suitable cross-linking methods known to a person of skill may be used including both chemical and physical cross-linking methods.

In one embodiment of the present disclosure the polymer has been cross-linked by physical means, such as by dry heat. The dry heat treatment is usually performed at temperatures between 100° C. and 250° C., such as about 110° C. to about 200° C. In particular the temperature may be in the range of 110-160° C., e.g. in the range of 110-140° C., or in the range of 120-180° C., or in the range of 130-170° C., or in the range of 130-160° C., or in the range of 120-150° C. The period of time for cross-linking may be optimised by a skilled person and is normally a period between about 10 minutes to about 12 hours, such as about 1 hour to about 10 hours, for example between about 2 hours to about 10 hours, such as between about 4 hours to about 8 hours, for example between about 5 hours to about 7 hours, such as about 6 hours.

In another embodiment, the polymer has been cross-linked by chemical means, i.e. by exposure to a chemical cross-linking agent. Examples of suitable chemical cross-linking agents include but are not limited to aldehydes, in particular glutaraldehyde and formaldehyde, acyl azide, carbodiimides, hexamethylene diisocyanate, polyether oxide, 1,4-butanedioldiglycidyl ether, tannic acid, aldose sugars, e.g. D-fructose, genipin and dye-mediated photo-oxidation. Specific compounds include but are not limited to 1-(3-dimethylaminopropyl)-3-ethylcarbodiimide hydrochloride (EDC) and dithiobis(propanoic dihydrazide) (DTP).

In one embodiment, the biocompatible polymer particles used for the preparation of the paste according to the present disclosure may be obtained from cross-linked sponges of e.g. gelatine or collagen, in particular cross-linked sponges of gelatine (such as the commercially available Spongostan® sponges and Surgifoam® sponges). The cross-linked sponges are micronized by methods known in the art to obtain a cross-linked biocompatible polymer in powder form, such as by rotary bed, extrusion, granulation and treatment in an intensive mixer, or milling (e.g. by using a hammer mill or a centrifugal mill). The cross-linked biocompatible polymer in powder form is then mixed with an amount of aqueous medium to provide a paste of a desirable consistency.

Spongostan®/Surgifoam® available from Ethicon is a gelatine based cross-linked absorbable haemostatic sponge. It absorbs >35 g of blood/g and within 4-6 weeks it is completely absorbed in the human body.

In one embodiment, the paste comprising a biocompatible polymer comprises cross-linked gelatine particles obtained from a micronized porous gelatine sponge, which has been cross-linked by dry heat treatment.

Micronized porous gelatine sponges may be prepared by mixing an amount of soluble gelatine with an aqueous medium in order to create a foam comprising a discontinuous gas phase, drying said foam and crosslinking the dried foam by exposure to dry heat. The obtained cross-linked sponge can be micronized by methods known in the art. The gelatine foam usually has a gelatine concentration from about 1% to 70% by weight, usually from 3% to 20% by weight. Drying is usually performed at about 20° C. to about 40° C. for about 5 to 20 hours. The dried foam is usually cross-linked by exposure to a temperature of about 110° C. to about 200° C. for about 15 minutes to about 8 hours, such as at about 150° C. to about 170° C. for about 5 to 7 hours.

In one embodiment, the biocompatible polymer particles used for the preparation of the paste according to the present disclosure are obtained from cross-linked gels of e.g. gelatine or collagen, in particular cross-linked gelatine gels. The cross-linked gels may be micronized as described above. The cross-linked biocompatible polymer in powder form is then mixed with an amount of aqueous medium to provide a paste of a desirable consistency.

In one embodiment, the paste comprising a biocompatible polymer comprises cross-linked gelatine particles obtained from a micronized gelatine gel, which has been cross-linked by dry heat treatment.

In one embodiment, the paste comprising a biocompatible polymer comprises or consists of cross-linked gelatine particles obtained from a gelatine hydrogel. A gelatine hydrogel may be prepared by dissolving an amount of gelatine in an aqueous buffer to form a non-cross-linked hydrogel, typically having a solids content from 1% to 70% by weight, usually from 3% to 10% by weight. The gelatine may be cross-linked, for example by exposure to either glutaraldehyde (e.g. 0.01% to 0.05% w/w, overnight at 0° C. to 15° C. in aqueous buffer), sodium periodate (e.g. 0.05 M, held at 0° C. to 15° C. for 48 hours) or 1-ethyl-3-(3-dimethylaminopropyl) carbodiimide (EDC) (e.g. 0.5% to 1.5% w/w, overnight at room temperature), or by exposure to about 0.3 to 3 megarads of gamma or electron beam radiation. The resulting crosslinked hydrogels may be fragmented and dried to obtain a gelatine powder. Alternatively, gelatine particles can be suspended in an alcohol, preferably methyl alcohol or ethyl alcohol, at a solids content of 1% to 70% by weight, usually 3% to 10% by weight, and cross-linked by exposure to a cross-linking agent, typically glutaraldehyde (e.g., 0.01% to 0.1% w/w, overnight at room temperature). When cross-linking with glutaraldehyde, the cross-links are formed via Schiff bases which may be stabilized by subsequent reduction, e.g. by treatment with sodium borohydride. After cross-linking, the resulting granules may be washed in water and optionally rinsed in an alcohol and dried to obtain a gelatine powder. In one embodiment, cross-linked gelatine particles are prepared essentially as described in U.S. Pat. No. 6,066,325.

In one embodiment, the paste comprises a biocompatible polymer in a content of about 7% to 20%, such as about 8% to 18%, for example about 10% to 16%, such as about 11%-15%, for example about 12%-14%.

In one embodiment, the paste comprises a biocompatible polymer in a content of about 7% to 20%, such as about 7% to 18%, for example about 7% to 16%, such as about 7% to 14%, for example about 7% to 13%, such as about 7% to 12%, for example about 7% to 11%, such as about 7% to 10%, for example about 7% to 9%.

In one embodiment, the paste comprises a biocompatible polymer in a content of about 7% to 20%, such as about 10% to 20%, for example about 11% to 20%, such as about 12% to 20%, for example about 13% to 20%, such as about 14% to 20%, for example about 15% to 20%, such as about 17% to 20%, for example about 19% to 20%.

In one embodiment, the paste comprises a biocompatible polymer in a content of about 10% to 20%, such as about 10% to 18%, for example about 10% to 16%, such as about 10% to 15%.

The paste comprising a biocompatible polymer may further comprise one or more hydrophilic agent(s), such as for example one or more polyol(s) and/or one or more poly(ethylene glycol) (s) (PEG).

In one embodiment, the paste comprising a biocompatible polymer comprises one or more further bioactive agent(s). Such one or more bioactive agent(s) may be able to stimulate haemostasis, wound healing, bone healing, tissue healing and/or tendon healing.

In one embodiment, the paste comprising a biocompatible polymer comprises one or more extrusion enhancer(s), such as for example albumin.

In one embodiment, the paste comprising a biocompatible polymer comprises one or more antimicrobial agents, such as one or more antibacterial agents.

In one embodiment, the paste comprising a biocompatible polymer comprises benzalkonium chloride.

The paste comprising a biocompatible polymer as described herein may be prepared according to techniques known in the art. Accordingly, the paste comprising a biocompatible polymer may be prepared by mixing of a biocompatible polymer powder with an aqueous medium to generate said paste.

The paste comprising a biocompatible polymer is usually provided in sterile form. Thus, in one embodiment the paste comprising a biocompatible polymer is sterile.

The paste comprising a biocompatible polymer further comprises an aqueous medium, such as for example water, saline, a calcium chloride solution or a buffered aqueous medium.

### Aqueous Medium

An aqueous medium may be used in the present disclosure for preparing the paste comprising a biocompatible polymer.

The aqueous medium of the present disclosure may be any aqueous medium suitable for preparing a paste known to a person of skill, e.g. water, saline or a buffered aqueous medium. The water may be WFI (Water For Injection). It is important that the aqueous medium is selected so that the reconstituted paste product is essentially isotonic when intended for use on a human or animal subject, such as for haemostatic and/or wound healing purposes. The aqueous medium is preferably sterile.

The aqueous medium of the present disclosure is in one embodiment a saline solution.

In one embodiment, the aqueous medium is a calcium chloride solution.

In other embodiments, the aqueous medium is water.

The aqueous medium may also be a buffered aqueous medium suitable for use in a haemostatic paste. Any suitable buffering agent known to a person of skill may be used, such as one or more buffering agents selected from the group consisting of: Sodium citrate; Citric acid, Sodium citrate; Acetic acid, Sodium acetate; K₂HPO₄, KH₂PO₄; Na₂HPO₄, NaH₂PO₄; CHES; Borax, Sodium hydroxide; TAPS; Bicine; Tris; Tricine; TAPSO; HEPES; TES; MOPS; PIPES; Cacodylate; SSC; MES, or others. The pH of the buffered aqueous medium should be suitable for creating a haemostatic paste intended for human use and can be determined by the skilled person.

Thus, in one embodiment the paste comprising a biocompatible polymer comprises an aqueous medium selected from the group consisting of water, saline, a calcium chloride solution and a buffered aqueous medium.

In one embodiment, the paste comprising a biocompatible polymer comprises between about 60% to about 95% of water, for example about 70% to about 90% of water, such as between about 75% to about 90% of water, for example between about 80% to about 90% of water.

The aqueous medium may comprise one or more hydrophilic agent(s), such as for example one or more polyol(s) or one or more poly(ethylene glycol) (s) (PEG).

In one embodiment, the aqueous medium comprises one or more further bioactive agent(s). Such one or more bioactive agent(s) may be able to stimulate haemostasis, wound healing, bone healing, tissue healing and/or tendon healing.

In one embodiment, the aqueous medium comprises one or more extrusion enhancer(s), such as for example albumin.

### Hydrophilic Compounds

In one embodiment, the haemostatic composition comprises one or more hydrophilic compounds. Hydrophilic compounds usually contain polar or charged functional groups, rendering them soluble in water. Inclusion of one or more hydrophilic compounds in the haemostatic composition of the present disclosure is believed to have a beneficial effect on thrombin stability and may improve reconstitution efficiency of the dry thrombin composition. Hydrophilic compounds may also improve consistency of the haemostatic composition.

In one embodiment, the hydrophilic compound is a hydrophilic polymer. The hydrophilic polymer may be natural or synthetic, linear or branched, and have any suitable length.

In one embodiment, the hydrophilic polymer is selected from the group consisting of Polyethylenimine (PEI), Poly(ethylene glycol) (PEG), Poly(ethylene oxide), Poly(vinyl alcohol) (PVA), Poly(styrenesulfonate) (PSS), Poly(acrylic acid) (PAA), Poly(allylamine hydrochloride) and Poly(vinyl acid). In one embodiment, the hydrophilic compound is PEG.

In one embodiment, the hydrophilic compound is selected from the group consisting of Cetylpyridinium Chloride, Docusate Sodium, Glycine, Hypromellose, Phthalate, Lechitin, Phospholipids, Poloxamer, Polyoxyethylene Alkyl Ethers, Polyoxyethylene Castor Oil Derivatives, Polyoxyethylene Sorbitan Fatty Acid Esters, Polyoxyethylene Stearates, Polyvinyl Alcohol, Sodium Lauryl Sulfate, Sorbitan Esters (Sorbitan Fatty Acid Esters) and Tricaprylin.

In a preferred embodiment, the hydrophilic compound is a polyol. Thus, according to one embodiment of the invention, one or more polyols may be included in the haemostatic composition. Polyols may enhance the reconstitution rate of the dry thrombin composition, stabilize thrombin activity and play a role in ensuring an optimal consistency of the haemostatic composition.

A polyol as defined herein is a compound with multiple hydroxyl functional groups. Polyols include sugars (mono-, di-and polysaccharides), sugar alcohols and derivatives thereof. Especially preferred are sugar alcohols.

Monosaccharides include but are not limited to glucose, fructose, galactose, xylose and ribose.

Disaccharides include but are not limited to sucrose (saccharose), lactulose, lactose, maltose, trehalose and cellobiose.

Polysaccharides include but are not limited to starch, glycogen, cellulose and chitin.

A sugar alcohol, also known as a polyalcohol is a hydrogenated form of carbohydrate, whose carbonyl group (aldehyde or ketone, reducing sugar) has been reduced to a primary or secondary hydroxyl group (hence the alcohol). Sugar alcohols have the general formula H (HCHO) n+1H, whereas sugars have H (HCHO),HCO. Some common sugar alcohols which may be used in the method of the present disclosure include but are not limited to: Glycol (2-carbon), Glycerol (3-carbon), Erythritol (4-carbon), Threitol (4-carbon), Arabitol (5-carbon), Xylitol (5-carbon), Ribitol (5-carbon), Mannitol (6-carbon), Sorbitol (6-carbon), Dulcitol (6-carbon), Fucitol (6-carbon), Iditol (6-carbon), Inositol (6-carbon; a cyclic sugar alcohol), volemitol (7-carbon), Isomalt (12-carbon), Maltitol (12-carbon), Lactitol (12-carbon), Polyglycitol.

In one embodiment, the haemostatic composition comprises a single hydrophilic compound, such as a single polyol.

In one embodiment of the invention, the haemostatic composition comprises more than one hydrophilic compound, such as two, three, four, five, six or even more different hydrophilic compounds.

In a preferred embodiment, the hydrophilic compound is a polyol.

In one embodiment of the invention, the haemostatic composition comprises two polyols, for example mannitol and glycerol or trehalose and a glycol.

In one embodiment of the invention, the haemostatic composition comprises one or more sugar alcohols, such as one or more sugar alcohols selected from the group consisting of Glycol, Glycerol, Erythritol, Threitol, Arabitol, Xylitol, Ribitol, Mannitol, Sorbitol, Dulcitol, Fucitol, Iditol, Inositol, volemitol, Isomalt, Maltitol, Lactitol, Polyglycitol.

In one embodiment, the haemostatic composition comprises one or more sugar alcohols and one or more sugars, such as one sugar alcohol and one sugar.

In one embodiment, the haemostatic composition comprises one sugar alcohol and optionally one or more additional hydrophilic compounds, such as one or more polyols, which may be either sugar alcohols or sugars.

In one embodiment, the haemostatic composition does not comprise a sugar as the only polyol.

In one embodiment of the invention, the haemostatic composition comprises mannitol.

In one embodiment of the invention, the haemostatic composition comprises sorbitol.

In one embodiment of the invention, the haemostatic composition comprises glycerol.

In one embodiment of the invention, the haemostatic composition comprises trehalose.

In one embodiment of the invention, the haemostatic composition comprises glycol, such as propylene glycol.

In one embodiment of the invention, the haemostatic composition comprises xylitol.

In one embodiment of the invention, the haemostatic composition comprises maltitol.

In one embodiment of the invention, the haemostatic composition comprises sorbitol.

In one embodiment the haemostatic composition comprises from about 1% to about 20% of one or more hydrophilic compounds, for example from about 1% to about 15% of one or more hydrophilic compounds, such as from about 1% to about 10% of one or more hydrophilic compounds, for example from about 1% to about 7% of one or more hydrophilic compounds, such as from about 1% to about 5% of one or more hydrophilic compounds, such as from about 2% to about 5% of one or more hydrophilic compounds, for example from about 3% to about 5% of one or more hydrophilic compounds.

In one embodiment the hydrophilic compound of the present disclosure is not poly(ethylene glycol) (PEG).

The hydrophilic compound may be a component of the dry thrombin composition, a component of the paste comprising a biocompatible polymer and/or be incorporated into the haemostatic composition in a separate step after reconstitution of the dry thrombin composition.

In one embodiment, the one or more hydrophilic compound(s) is a component of the dry thrombin composition.

In one embodiment, the one or more hydrophilic compound(s) is a component of the paste comprising a biocompatible polymer.

In one embodiment, the one or more hydrophilic compound(s) is incorporated into the haemostatic composition in a separate step after reconstitution of the dry thrombin composition.

Preferably, the hydrophilic compound(s) is included in either the thrombin component or the paste component to avoid further mixing steps to produce the haemostatic composition.

### Further Bioactive Agents

In one embodiment of the invention, the haemostatic composition comprises one or more further bioactive agents capable of stimulating haemostasis, wound healing, bone healing, tissue healing and/or tendon healing. The one or more bioactive agents may be a component of the dry thrombin composition, the paste comprising a biocompatible polymer and/or be incorporated into the haemostatic composition in a separate step after reconstitution of the dry thrombin composition. Preferably, such bioactive agents are included in either the dry thrombin composition or in the paste comprising a biocompatible polymer to avoid further mixing steps. It is essential that the bioactive agent retains its bioactivity during storage and reconstitution, i.e. that the agent has retained its biological function in the final haemostatic composition. Many bioactive agents are unstable in solution, particularly enzymes and other proteins that may be degraded or lose their secondary structure when water is present.

The one or more further bioactive agents can be e.g. fibrinogen, fibrinogen in combination with Factor XIII, or fibrinogen and Factor XIII in combination with tranexamic acid.

In one embodiment, the haemostatic composition comprises one or more further bioactive agents that stimulate bone and/or tendon and/or tissue healing such as one or more growth factors selected from the group consisting of matrix metalloproteinases (MMPs), insulin-like growth factor 1 (IGF-I), platelet-derived growth factor (PDGF), vascular endothelial growth factor (VEGF), basic fibroblast growth factor (bFGF) and transforming growth factor beta (TGF-β).

In one embodiment, the haemostatic composition comprises one or more Bone Morphogenetic Proteins (BMPs). Bone morphogenetic proteins (BMPs) are a subgroup of the TGF-β superfamily. Bone Morphogenetic Proteins (BMPs) are a group of growth factors also known as cytokines and as metabologens. Originally discovered by their ability to induce the formation of bone and cartilage, BMPs are now considered to constitute a group of pivotal morphogenetic signals, orchestrating tissue architecture throughout the body.

In one embodiment, the haemostatic composition comprises one or more matrix metalloproteinases (MMPs). MMPs are zinc-dependent endopeptidases. MMPs have a very important role in the degradation and remodeling of the extracellular matrix (ECM) during the healing process after an injury. Certain MMPs including MMP-1, MMP-2, MMP-8, MMP-13, and MMP-14 have collagenase activity, meaning that, unlike many other enzymes, they are capable of degrading collagen I fibrils.

These growth factors all have different roles during the healing process. IGF-1 increases collagen and proteoglycan production during the first stage of inflammation, and PDGF is also present during the early stages after injury and promotes the synthesis of other growth factors along with the synthesis of DNA and the proliferation of cells. The three isoforms of TGF-β (TGF-β1, TGF-β2, TGF-β3) are known to play a role in wound healing and scar formation. VEGF is well known to promote angiogenesis and to induce endothelial cell proliferation and migration.

In one embodiment, the haemostatic composition of the present disclosure comprises flakes or particles of extracelluar matrix (ECM). ECM is the extracellular part of animal tissue that usually provides structural support to the animal cells in addition to performing various other important functions. ECM has been shown to have very beneficial effect in healing as it facilitates functional tissue regeneration.

The variety of further bioactive agents that can be used in conjunction with the haemostatic composition of the invention is vast. In general, bioactive agents which may be administered via the haemostatic composition of the invention include, without limitation, antiinfectives, such as antibiotics and antiviral agents; analgesics and analgesic combinations; antihelmintics; antiarthritics; anticonvulsants; antidepressants; antihistamines; antiinflammatory agents; antimigraine preparations; antineoplastics; antiparkinsonism drugs; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators, including general coronary, peripheral and cerebral; central nervous system stimulants; hormones, such as estradiol and other steroids, including corticosteroids; immunosuppressives; muscle relaxants; parasympatholytics; psychostimulants; naturally derived or genetically engineered proteins, polysaccharides, glycoproteins, or lipoproteins; oligonucleotides, antibodies, antigens, cholinergics, chemotherapeutics, radioactive agents, osteoinductive agents, cystostatics heparin neutralizers, procoagulants and haemostatic agents, such as prothrombin, thrombin, fibrinogen, fibrin, fibronectin, heparinase, Factor X/Xa, Factor VII/VIla, Factor VIII/VIIIa, Factor IX/IXa, Factor XI/XIa, Factor XII/XIIa, Factor XIII/XIIIa, tissue factor, batroxobin, ancrod, ecarin, von Willebrand Factor, platelet surface glycoproteins, vasopressin, vasopressin analogs, epinephrine, selectin, procoagulant venom, plasminogen activator inhibitor, platelet activating agents and synthetic peptides having haemostatic activity.

In one embodiment, the one or more further bioactive agent(s) is a component of the dry thrombin composition.

In one embodiment, the one or more further bioactive agent(s) is a component of the paste comprising a biocompatible polymer.

In one embodiment, the one or more further bioactive agent(s) is incorporated into the haemostatic composition in a separate step after reconstitution of the dry thrombin composition.

### Further Compounds

The haemostatic composition of the invention may further comprise one or more of the following: DMSO (dimethyl sulfoxide) and/or 2-Methyl-2,4-pentanediol (MPD).

In one embodiment, the haemostatic composition of the present disclosure comprises one or more antimicrobial agents, such as one or more antibacterial agents.

In one embodiment, the haemostatic composition of the present disclosure comprises benzalkonium chloride.

In one embodiment, the haemostatic composition of the present disclosure does not comprise an antimicrobial agent.

In one embodiment, the haemostatic composition further comprises an extrusion enhancer, i.e. a compound capable of facilitating extrusion of a paste from a syringe.

It has previously been shown that the provision of certain extrusion enhancers, such as albumin in an appropriate amount, enables the use of higher gelatine concentrations as it decreases the amount of force needed to extrude the gelatine paste composition from e.g. a syringe. The use of higher gelatine concentrations may in turn improve the haemostatic properties of such products. It is necessary to provide the extrusion enhancers in appropriate amounts. The amounts are preferably high enough so as to obtain the extrusion effect, i.e. to enable a flowable paste even for relatively high amounts of the biocompatible polymer, e.g. cross-linked gelatine, so that the haemostatic composition can be accurately applied by a surgeon using e.g. a syringe comprising an applicator tip; on the other hand, the amounts shall be as low as to prevent potential negative functional properties of the haemostatic composition.

The extrusion enhancer is preferably albumin, especially human serum albumin.

In the haemostatic composition of the present invention, the extrusion enhancer, such as albumin, is present in an amount of between about 0.1% to about 10%, such as between about 0.2% to about 8%, for example between about 0.3% to about 7%, preferably between about 0.5% to about 5%, such as between about 1% to about 4%.

In one embodiment, the haemostatic composition of the present invention comprises trace amounts of albumin, such as less than 0.1%, for example less than 0.01%, such as less than 0.001%, for example less than 0.0001%.

The one or more further compound(s) may be a component of the dry thrombin composition, a component of the paste comprising a biocompatible polymer and/or be incorporated into the haemostatic composition in a separate step after reconstitution of the dry thrombin composition.

In one embodiment, the one or more further compound(s) is a component of the dry thrombin composition.

In one embodiment, the one or more further compound(s) is a component of the paste comprising a biocompatible polymer.

In one embodiment, the one or more further compound(s) is incorporated into the haemostatic composition in a separate step after reconstitution of the dry thrombin composition.

### Making the Haemostatic Composition/Reconstitution of Dry Thrombin Composition

The present disclosure relates to a method for reconstituting a dry thrombin composition directly in a paste to generate a haemostatic composition suitable for use in the treatment of a wound; particularly for haemostatic purposes.

Thus, in one embodiment, a method of preparing a haemostatic composition is provided, the method comprising the steps of:
a) providing a dry thrombin composition in a first container;
b) providing a paste comprising a biocompatible polymer in a second container;
c) connecting the first container and the second container using suitable connecting means; and
d) mixing the contents of the containers

In one embodiment, a method for reconstituting a dry thrombin composition is provided, the method comprising the steps of:
a) providing a dry thrombin composition in a first container;
b) providing a paste comprising a biocompatible polymer in a second container;
c) connecting the first container and the second container using suitable connecting means; and
d) mixing the contents of the containers.

The containers are usually syringes, more preferably interconnectable syringes.

In one embodiment, the suitable connecting means comprises a static mixer. Said static mixer may provide the ability to mix air into the paste during mixing. The dimensions of the static mixer may influence the consistency of the haemostatic composition and the ability to mix air into the haemostatic composition.

In one embodiment, the suitable connecting means comprises a connector portion of a standard type, such as a Luer lock or Luer slip connector. The dimensions of the Luer lock or Luer slip connection may influence the consistency of the haemostatic composition and the ability to mix air into the haemostatic composition.

The mixing is performed by transferring the content of the containers back and forth a number of times.

In one embodiment, the number of transfers is less than 20 times, for example less than 15 times, such as less than 12 times, for example less than 10 times, such as less than 6 times.

In one embodiment, the number of transfers is less than 10, for example less than 9, such as less than 8 times, for example less than 7 times, such as less than 6 times, for example less than 5 times.

In one embodiment, the number of transfers is less than 8.

In a preferred embodiment, the number of transfers is about 6 times or less.

Mixing with 6 transfers can be performed as follows: The mixing is initiated by transfer of the paste comprising a biocompatible polymer into the first container holding the dry thrombin composition. Thus, the first transfer is characterized in the transfer of the paste comprising a biocompatible polymer from the second container into the first container. The second transfer is characterized in the transfer of the thrombin and paste mixture, resulting from the first transfer, from the first container into the second container, initially holding the paste comprising a biocompatible polymer. The third transfer is characterized in the transfer of the thrombin and paste mixture, resulting from the second transfer, from the second container into the first container. The fourth transfer is characterized in the transfer of the thrombin and paste mixture, resulting from the third transfer, from the first container into the second container. The fifth transfer is characterized in the transfer of the thrombin and paste mixture, resulting from the fourth transfer, from the second container into the first container. The sixth and final transfer is characterized in the transfer of the thrombin and paste mixture, resulting from the fifth transfer, from the first container into the second container.

In one embodiment, more than six transfers between the first and second containers are used for reconstituting the dry thrombin composition in the paste.

In one embodiment, the final transfer is characterized in the transfer of the thrombin and paste mixture from the first container into the second container. The resulting haemostatic composition may then be applied directly from said second container onto a site of treatment, e.g. onto a bleeding wound. An applicator tip may in some embodiments be fitted to the syringe and employed as described previously.

The mixing of the dry thrombin composition and the paste comprising a biocompatible polymer result in substantially homogenous distribution of thrombin in the haemostatic composition.

### The Haemostatic Composition

The haemostatic composition of this disclosure is prepared by reconstituting a dry thrombin composition directly in a paste comprising a biocompatible polymer. The reconstitution of the paste may be performed by methods as described herein. The haemostatic composition has a substantially homogenous distribution of thrombin in the haemostatic composition.

In one embodiment, the homogenous distribution of thrombin is characterized by a variation in thrombin content throughout the haemostatic composition of less than 20%, for example less than 10%, such as less than 5%, for example less than 4%, such as less than 3%, for example less than 2%, such as less than 1%. The variation in thrombin content may be measured as difference in thrombin activity or concentration between different fractions of the haemostatic paste in the container, such as for example between the start fraction of a syringe, the mid fraction of a syringe or the end fraction of a syringe.

In one embodiment the variation in thrombin content throughout the haemostatic composition is less than 10%.

In one embodiment the variation in thrombin content throughout the haemostatic composition is less than 5%.

In one embodiment the variation in thrombin content throughout the haemostatic composition is less than 4%.

In one embodiment the variation in thrombin content throughout the haemostatic composition is less than 3%.

In one embodiment the variation in thrombin content throughout the haemostatic composition is less than 2%.

In one embodiment the variation in thrombin content throughout the haemostatic composition is less than 1%.

The haemostatic composition obtained by the methods described herein is preferably a flowable composition. The haemostatic composition obtained by the methods described herein is suitable for use in haemostasis and/or wound healing.

The haemostatic composition may comprise one or more hydrophilic agent(s), such as for example one or more polyol(s) or one or more poly(ethylene glycol) (s) (PEG).

In one embodiment, the haemostatic composition comprises one or more further bioactive agent(s) besides thrombin. Such one or more bioactive agent(s) may be able to stimulate haemostasis, wound healing, bone healing, tissue healing and/or tendon healing.

In one embodiment, the haemostatic composition comprises one or more extrusion enhancer(s), such as for example albumin.

The haemostatic composition may be obtained by any method of reconstitution of a dry thrombin composition as described herein.

In one embodiment, the haemostatic composition has a consistency within the range of about 100 g ×sec to about 10,000 g ×sec, such as from about 500 g ×sec to about 5000 g ×sec, for example from about 1000 g ×sec to about 3000 g ×sec, such as from about 1500 g ×sec to about 2000 g ×sec.

In one embodiment, the haemostatic composition has a consistency of less than about 5000 g ×sec, for example less than about 4000 g ×sec, such as less than about 3000 g ×sec, for example less than about 2000 g ×sec.

### Containers

Any suitable containers known to a person of skill may be used for preparing the haemostatic composition, such as vials, jars, tubes, trays, cartridges or syringes.

The dry thrombin composition is provided in a first container and the paste comprising a biocompatible polymer is provided in a second container.

The first and the second containers may be made from any suitable material such as plastic, glass, ceramic, plastic or metal, such as stainless steel. Examples of suitable plastic materials include but are not limited to polyethylene, polypropylene, polystyrene, polyvinyl chloride, and polytetrafluoroethylene (PTFE).

In one embodiment, the dry thrombin composition is provided in a first container which may be selected from a syringe, a vial, a jar, a tube, a tray, or a cartridge.

In a preferred embodiment said first container holding the dry thrombin composition is a medical delivery device suitable for dispensing flowable haemostatic compositions to a patient in need thereof, such as a syringe.

The first container is usually made from a material suitable for chemical surface sterilisation without influencing the content of the container. For example, said first container may be made from a material which is impermeable to ethylene oxide, such as for example made from metal, glass or a plastic which is impermeable to ethylene oxide.

The dry thrombin composition may preferably be provided in a glass container, thus allowing sterilisation of said container using ethylene oxide gas. In one embodiment, said first container is a glass syringe. In one embodiment, said first container is a syringe having a glass insert holding the dry thrombin.

In one embodiment, the paste comprising a biocompatible polymer is provided in a second container which may be selected from a syringe, a vial, a jar, a tube, a tray, or a cartridge. The paste comprising a biocompatible polymer may be prepared by methods known in the art, such as by mixing of a biocompatible polymer powder with an aqueous medium to generate said paste. The paste comprising a biocompatible polymer may suitably be prepared in bulk and transferred/aliquoted into said second container.

In a preferred embodiment the container holding the paste comprising a biocompatible polymer is a medical delivery device suitable for dispensing flowable haemostatic compositions to a patient in need thereof, such as a syringe. In one embodiment, the second container is a single-use plastic syringe . . .

In one embodiment, the first and second containers are interconnectable. The connector portion may be a connector portion of a standard type, such as a Luer lock or Luer slip connector. The connector portion may be provided with a threaded portion for secure connection with matching connector. The dimensions of said Luer lock or Luer slip connection may be able to change the ability of mixing in air into the haemostatic composition during mixing of the dry thrombin composition and the paste comprising a biocompatible polymer. Further, the dimensions of the Luer lock or Luer slip connection may be able to influence the consistency of the haemostatic composition.

In one embodiment, the connector portion comprises a static mixer. The dimensions of said static mixer may be able to change the ability of mixing in air into the haemostatic composition during mixing of the dry thrombin composition and the paste comprising a biocompatible polymer. Further, the dimensions of the static mixer may be able to influence the consistency of the haemostatic composition.

### Outer Package

In one embodiment the dry thrombin composition and/or the paste comprising a biocompatible polymer contained within e.g. a syringe, such as the herein disclosed syringe, or other containment unit, is further contained within an outer package so that the product is kept sterile until use. This will allow the user to remove the outer package and transfer the components of the haemostatic composition into a sterile field.

The outer package is usually made from a flexible, semi-rigid or rigid material and typically consists of materials such as plastic, aluminium foil and/or plastic laminate, where the plastic may be selected from the group consisting of PET, PETG, PE, LLDPE, CPP, PA, PETP, METPET, Tyvek and optionally bonded with an adhesive, such as polyurethane, or co-extruded.

In one embodiment, the outer package is an aluminium foil outer package.

The outer package preferably forms a complete barrier to moisture.

The outer package is preferably able to endure sterilisation treatment such as by radiation.

In one embodiment, the first syringe comprising a dry thrombin composition and the second syringe comprising a paste comprising a biocompatible polymer are contained in separate outer packages.

### Sterilisation

The dry thrombin composition, the paste comprising a biocompatible polymer and/or the haemostatic composition of the present disclosure are preferably sterile. Any suitable sterilisation technique known in the art may be utilised. Sterilisation refers to any process that effectively kills or eliminates transmissible agents (such as fungi, bacteria, viruses, prions and spore forms etc.). Sterilisation can be achieved through e.g. application of heat, chemicals, and/or irradiation.

Sterilisation may be achieved by heat sterilisation, include autoclaving (uses steam at high temperatures) and dry heat.

Sterilisation may be achieved by irradiation, e.g. ionizing irradiation, so as to provide sterility to the components. Such irradiation may include e-beam (beta irradiation), X-rays, gamma and beta rays, UV light and subatomic particles. The level of irradiation and conditions for sterilisation, including the time, are those that provide sterile compositions. Sterilisation conditions are similar to those currently utilized in the art and can be determined by the skilled person.

Sterilisation may be performed by chemical sterilisation such as by using ethylene oxide gas, ozone, chlorine bleach, glutaraldehyde, formaldehyde, ortho phthalaldehyde, hydrogen peroxide and/or peracetic acid.

The dry thrombin composition is usually prepared using aseptic methods thereby providing a sterile dry thrombin composition in said first container.

In one embodiment, the surface of the first container comprising a dry thrombin composition is sterilised with chemical sterilisation, such as with ethylene oxide gas.

In one embodiment, the paste comprising a biocompatible polymer is sterilised by irradiation, such as by gamma irradiation.

In one embodiment, sterilisation of the second container comprising a paste comprising a biocompatible polymer is sterilised using beta or gamma irradiation, thereby providing a sterile paste and a sterile container.

In one embodiment sterilisation of the second container comprising a paste comprising a biocompatible polymer occurs as terminal sterilisation, i.e. when the second container comprising a paste comprising a biocompatible polymer is contained within an outer package.

### Medical Use

The present disclosure further relates to use of the haemostatic composition obtained by the methods of this disclosure for promoting haemostasis and/or wound healing.

The haemostatic composition of the present disclosure may e.g. be used in an array of surgical procedures wherein bleeding control is desired. The haemostatic composition is in the form of a paste which conforms to irregular surfaces to stop bleeding fast and it is therefore useful for providing rapid haemostasis on rough or uneven surfaces where haemostatic sponges are not efficient.

In general, haemostatic pastes are prepared directly at the surgical site at the time of need by the medical practitioner, i.e. the doctors or nurses usually by addition of liquid (optionally comprising thrombin) to a container, such as a syringe, containing an amount of a biocompatible polymer. The biocompatible polymer may be pre-wetted with the liquid or be essentially dry (free-flowing powder). The paste is thus often prepared under extremely stressful conditions and it is therefore essential that the process for preparing the paste is simple and fast to ensure that the bleeding is arrested as quickly as possible and that no mistakes are made while preparing the paste such that the nurse can keep focus on the needs of the surgeon instead of on preparing the haemostat. It is also important that the consistency of the paste is suitable for use as a haemostatic paste and that the consistency of the product is independent from preparation to preparation and over time.

Currently available flowable paste products (Floseal® and Surgiflo®) require reconstitution of a thrombin composition in a liquid prior to mechanical mixing of said reconstituted thrombin solution with the biocompatible polymer by passing the biocompatible polymer and the liquid between two connected syringes a number of times to obtain a substantially homogenous paste. The reconstitution of the thrombin is time-consuming and error prone, two undesired factors in an OR setting. These products are often pre-prepared in the OR before surgery in case they are needed under surgery and unused product is often discarded causing unnecessary high OR costs.

The method of preparing a haemostatic composition of the present disclosure is superior to the currently available methods as it reduces number of handling steps in the procedure by allowing direct reconstitution of a dry thrombin composition in a paste. The haemostatic composition of the present disclosure may be prepared simply by adding an amount of a paste comprising a biocompatible polymer to a container comprising the dry thrombin composition and mixing the content, such as transferring the content between two interconnected syringes a number of times, whereupon a ready-to-use haemostatic paste comprising substantially homogenously distributed thrombin is formed.

The fact that no prior reconstitution of the dry thrombin composition in a liquid is required also means that less time is spent preparing the paste, which in turn leads to increased patient safety, both due to the fact that the haemostatic composition can be applied to the patient faster and that the simple preparation method decreases the likelihood of mistakes being made during the preparation of the haemostatic composition. Also, the method of preparing a haemostatic composition of the present disclosure can decrease OR costs as there is no need to pre-prepare the current product before surgery since preparation is so simple and fast.

Another notable advantage of the method of the present invention is that a kit consisting of fewer components can be prepared as compared to current haemostatic flowable kits. All there is required to prepare a flowable paste composition in the OR is the dry thrombin composition as described herein comprised within a first container, such as a syringe and a second container, such as a medical delivery device comprising a paste comprising a biocompatible polymer. Upon connection of the two and mixing, a ready-to-use flowable paste containing all necessary agents for effective haemostasis including thrombin is formed. Thus, no extra syringes, vial adapters, needles and mixing bowls are required with the product prepared according to the methods of the present disclosure. This means that the manufacturing costs can be decreased and also ensures good patient safety, since there are less components for the OR staff to keep track of during surgery. Needle-free preparation of the haemostat also ensures the safety of the OR staff.

In one embodiment the present disclosure relates to a method for arresting bleeding/promoting haemostasis in an individual in need thereof by application of the haemostatic composition prepared by the methods of the present disclosure to a site of bleeding.

The haemostatic composition of the present disclosure may be used for any type of surgery including general surgery, cardiothoracic surgery, vascular surgery, plastic surgery, paediatric surgery, colorectal surgery, transplant surgery, surgical oncology, trauma surgery, endocrine surgery, breast surgery, skin surgery, otolaryngology, gynaecology, oral and maxillofacial surgery, dental Surgery, orthopaedic surgery, neurosurgery, ophthalmology, podiatric surgery, urology.

In one embodiment the present disclosure relates to a method for promoting wound healing in an individual in need thereof by application of the haemostatic composition prepared by the methods of the present disclosure to the wound.

A “wound” refers broadly to injuries to the skin and/or underlying (subcutaneous) tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum). The present disclosure relates to treatment of any type of wound mentioned above using the haemostatic composition of the present disclosure.

The treatment of a wound can in principle result in healing of the wound or in accelerated healing of the wound. The accelerated healing can be a result of e.g. administration of a wound-healing promoting substance. Alternatively, the wound healing can be promoted by preventing bacterial or viral infection, or by reducing the risk of such an infection which would otherwise have prolonged the wound treatment process.

In one embodiment the present disclosure relates to a method for promoting bone and/or tendon healing in an individual in need thereof by application of the haemostatic composition prepared by the methods of the present disclosure to the injured bone/tendon.

The “individual” referred to herein may be any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In one embodiment the present disclosure relates to a haemostatic composition as disclosed herein, for use in the treatment of a wound, e.g. for arresting bleeding or for promoting wound healing.

### a Haemostatic Kit

The present disclosure further relates to a haemostatic kit comprising the dry thrombin composition of the present disclosure and a paste comprising a biocompatible polymer matched to the amount of the dry thrombin composition so that upon mixing, a haemostatic composition having a thrombin content suitable for use in haemostasis will form.

Hence, in one embodiment the present disclosure relates to a haemostatic kit comprising:
a) a first syringe comprising a dry thrombin composition;
b) a second syringe comprising a paste comprising a biocompatible polymer; and
c) optionally an outer package, wherein the two syringes are interconnectable.

In one embodiment, the haemostatic kit comprises:
a) a first syringe comprising a dry thrombin composition in an outer package;
b) a second syringe comprising a paste comprising a biocompatible polymer in an outer package; and
c) optionally an outer package comprising the components of a) and b), wherein the two syringes are interconnectable.

In one embodiment, the kit further comprises one or more applicator tips.

The kit may optionally contain instructions for use of the kit.

The components of the haemostatic kit may be as described elsewhere herein.

### EXAMPLES

### Example 1: Thrombin Distribution in Paste

### Materials:

2000 IU dry thrombin in a 10 mL glass syringe (first syringe).

5 mL of a gelatine paste in a 10 ml syringe (second syringe).

### Method:

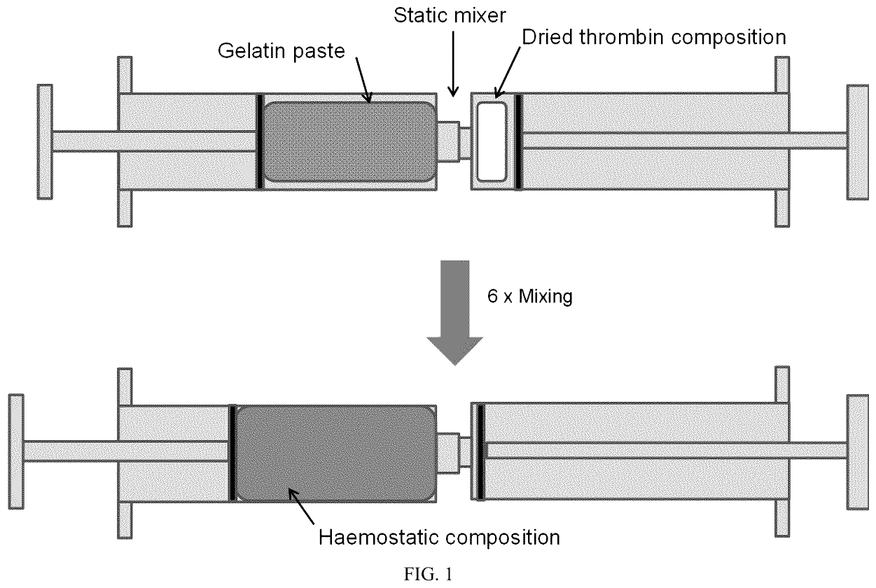

The two syringes are interconnected via a luer lock and the gelatine paste content is transferred to the syringe containing the dry thrombin composition (first transfer). The resulting mixture of thrombin and gelatine paste is then transferred back and forth between the first and the second syringes for a number of five times, resulting in a haemostatic composition contained in the second syringe (FIG. 1). The total number of transfers is 6 times. The haemostatic composition obtained is a flowable paste formulation comprising thrombin and is identified herein as the “6tomix” or “6TM” paste. As a consequence of mixing, a total of about 3 mL air is mixed into the paste providing a final volume of the paste of about 8 mL.

### Results:

The final haemostatic composition is a paste having a volume of about 8 mL.

The thrombin content of the haemostatic composition is measured as the mean thrombin activity in different fractions of the haemostatic composition.

The following fractional divisions are applied: The start fraction (i.e. the first ⅓ haemostatic composition extruded from the syringe), the mid fraction (the middle ⅓ haemostatic composition extruded from the syringe) and the end fraction (the last ⅓ haemostatic composition extruded from the syringe).

The thrombin activity of the total haemostatic composition is set at 100% and the mean thrombin activity of the individual fractions is calculated as percentage of the total activity. A completely homogenous thrombin distribution in the haemostatic composition would result in a mean thrombin activity of each of the three fractions of 33.333%.

| | Start fraction | Mid fraction | End fraction |
|---|---|---|---|
| Mean thrombin activity | 33% | 31% | 36% |

### Variation in Thrombin Content:

The variation in thrombin content is calculated as the percentage difference in mean thrombin activity between two fractions.

The maximal variation in thrombin content is here calculated as follows: (36/(31+36))−0.50)*100=3.7%.

In conclusion, this demonstrates that a dry thrombin composition can be reconstituted directly in a gelatine paste, via six transfers of the two components between two syringes, to generate a haemostatic composition having a substantially homogenous distribution of thrombin in the haemostatic composition.

### Example 2: Haemostatic Effect of Paste

### Purpose

The purpose of the study was to test the haemostatic efficacy of the 6TM paste of Example 1. The haemostatic efficacy of the paste was compared to that of the commercially available Surgiflo® paste. Surgiflo® is a sterile, absorbable porcine gelatine paste formulation approved for haemostatic use by applying to a bleeding surface and is an established product in the market.

Haemostatic efficacy was tested in a porcine spleen biopsy model as described below.

### Experimental Model

A porcine spleen biopsy-punch model was used applying 8 mm punctures (3 mm deep) in the spleen with an initial compression period of 10 seconds followed by an evaluation period of 120 seconds and following compression periods of 10 seconds.

The porcine spleen biopsy-punch model is an established model for evaluating haemostatic efficacy of haemostatic pastes in vivo (Hutchinson et al., 2015, Surgical Technology International XXVII). The porcine spleen biopsy-punch model of the present study is similar to the one used in Hutchinson et al., 2015. The present study was conducted with permission from the The Animal Experiments Inspectorate of Denmark (Dyreforsøgstilsynet).

### Experimental Animal

The pig is the animal of choice for this model since it has a large volume of blood (70 ml/kg) and a large vascular spleen that enables many haemostatic comparisons in a single animal. The female pigs of the present study had an approximate weight of 40 kg (±5 kg) and an approximate age of three months. The inclusion criterion for the pig was the weight. The lower weight limit was set to ensure that the organs to be tested were of an appropriate size, while the upper limit was a guidance to ensure that the size of the pig was standardized.

At the time of surgery, the tested pigs showed no signs of clinical illness.

### Anaesthesia and Fluid Therapy

The pigs had been fasting for at least 6 hours before surgery. The anaesthesia was induced by an intramuscular injection (1 mL/10 kg) of the following mixture: 6.25 mL Narcoxyl(Xylazin 20 mg/mL), 1.25 mL Ketaminol (Ketamin 100 mg/mL), 2 mL Turbogesic (Butorphanol 10 mg/mL), and 2 mL Metadon (Methadone 10 mg/mL) added to a vial of Zoletil 50 Vet (containing 125 mg Tiletamine and 125 mg Zolazepam).

The pigs were intubated and ventilated with a respirator with a mixture of 0.5 L oxygen/2.5 L air/min. The anaesthesia was maintained by intravenous administration of Fentanyl (50 μg/mL, 1 mL/10 kg/hour) and Propofol (10 mg/mL, 1.5 mL/kg/hour). The animals were kept normohydrated with Lactated Ringer's solution (125 mL/hour). It has previously been evaluated that the use of the mentioned medication does not affect haemostasis.

The pigs used in the present study were euthanized following surgery using an overdose of barbiturate resulting in cardiac arrest.

### Test Procedure

Sample preparation

The 6TM paste of the present invention was prepared as described in Example 1, i.e. by mixing a wet gelatine paste with a dry preparation of thrombin without prior reconstitution of the thrombin in saline.

The Surgiflo paste (control) was mixed with a thrombin solution according to the Surgiflo Instructions for Use dated 11 Jun. 2014.

Once mixed with thrombin, the chemical composition and water content of the control product (Surgiflo) and the 6TM paste of the present invention was the same.

To mimic storage conditions, the pastes were stored at 40° C. for 3 months prior to mixing with thrombin and subsequent testing, the only difference being that the control paste contained 2 ml less liquid (saline) than the 6TM paste of the present invention during storage. The 2 ml saline with thrombin were added to the control paste immediately prior to testing whereas the 6TM paste of the present invention was mixed with dry thrombin as described in Example 1 immediately prior to testing.

### Surgical Procedures

A midline abdominal incision was made to expose the spleen. An 8 mm punch (3 mm deep) was made in the spleen. The bleeding intensity was evaluated on a scale from 0-5 as described herein below. Only bleeding intensities 3 and 4 were regarded as acceptable. The punch was now ready for either a control sample or a test sample. A new punch was made for every test sample.

Each sample type was tested 7 times (n=7). The samples were tested in a randomized order.

A 12 minute negative control, using only wetted gauze, was performed at the initiation and completion of the testing period on each pig. The negative controls were used as an indication of the animal's ability to bleed throughout the study.

The primary test parameter was to measure the time to haemostasis (TTH). TTH is defined as the total time minus the final haemostasis evaluation period ensuring that no further bleeding occurred, i.e. no re-bleed.

The evaluation of bleeding intensity and the application of test samples and negative controls are described in detail below.

### Bleeding Intensity

The bleeding intensity of each punch was evaluated by the surgeon on a scale from 0-5 (see Table below).

Bleeding intensity was noted at t=0 for each punch. Only tests performed on wounds with bleeding intensity of 3-4 were used for further analysis.

TABLE
Bleeding intensity levels

| | |
|---|---|
| Level 0 | No bleeding (for at least 30 seconds) |
| Level 1 | No bleeding observed initially, bleeding observed |
| | within the first 30 seconds of injury |
| Level 2 | Bleeding observed immediately following injury, |
| | wound site fills in approximately 30 seconds |
| Level 3 | Bleeding observed immediately following injury, |
| | wound site fills in approximately 3 seconds |
| Level 4 | Bleeding observed immediately following injury, |
| | wound site fills immediately following injury |
| | (does not include arterial or pulsating bleeding) |
| Level 5 | Bleeding observed immediately following injury, |
| | wound site fills immediately following injury |
| | (including arterial or pulsating bleeding) |

### Negative Control

Wetted gauze was placed directly on the punch. Digital pressure was applied for 30 seconds followed by a 120 second haemostasis evaluation period. Haemostasis was evaluated (defined as no sieving of blood from under test article for 30 seconds). If haemostasis was not achieved within the 120 seconds, additional 30 seconds digital pressure was applied and a 120 second re-evaluation for haemostasis was performed. Tamponade application and observation periods were performed until bleeding stopped, and haemostasis achieved, or until the testing period reached 12 minutes. Haemostasis was not achieved within the 12 minutes testing period for the negative controls, thus showing the ability of the pig to bleed throughout the study.

### Application of Test Samples

Approximately 1-2 mL paste was applied directly into the punch with an applicator tip. During application the tip penetrated into the punch to ensure tissue contact. After application gauze wetted in 0.9% saline was placed on the punch. Digital pressure (tamponade) was applied for 10 seconds. The pressure was stopped and the gauze removed followed by evaluation of haemostasis. If no sieving of blood was seen from under the test article for 120 seconds, it was concluded that haemostasis was achieved and the experiment is ended. If blood sieved from under the test article in the 120 second time frame, the time for sieving was recorded and digital pressure was again applied for 10 seconds, after which haemostasis was inspected. This procedure was continued until haemostasis was achieved or for 12 minutes, whichever came first.

### Calculation Example for Evaluation of Time to Haemostasis

10 seconds of digital pressure, inspect for haemostasis: blood sieves after 39 seconds, digital pressure for another 10 seconds, inspect for haemostasis for 120 seconds: no sieving-conclusion: haemostasis was achieved after 10+39+10 seconds=59 sec. i.e. the last observation period is not included in calculating the TTH.

### Results

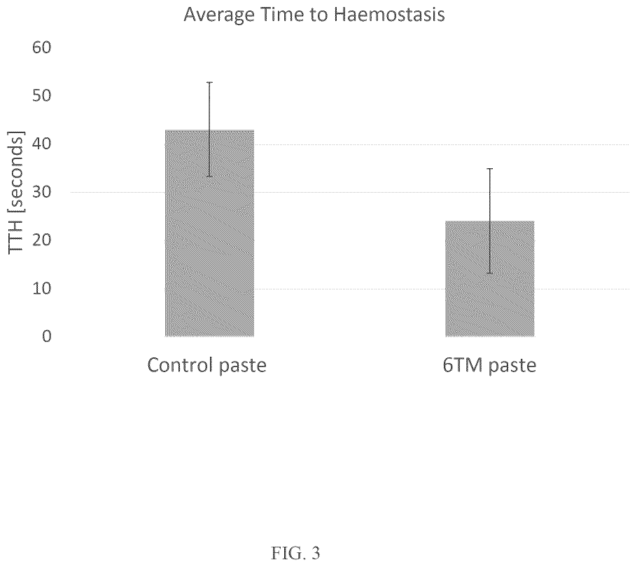

The results of the present study are shown in the table below and further depicted in FIG. 3.

| Measure | Control paste (n = 7) | 6TM paste (n = 7) |
|---|---|---|
| Time to Haemostasis | 43 ± 9.8 s | 24 ± 10.9 s |
| (TTH) | | |
| mean ± SEM* | | |

*SEM: Standard error of the mean.
“s” is seconds.

The results show an almost 2-fold reduction in the mean TTH of the 6TM paste as compared to the control paste.

The median TTH of the control paste was 37 seconds, while the median TTH of the 6TM paste was 10 seconds.

The data surprisingly shows that the 6TM paste prepared according to the present invention leads to haemostasis faster and more consistently than the control paste.

1. A method of preparing a haemostatic composition, the method comprising:
a) providing a dry thrombin composition in a first container;
b) providing a paste comprising a biocompatible polymer in a second container;
c) connecting the first container and the second container using suitable connecting means; and
d) mixing the contents of the containers.

2. The method according to claim 1, wherein the mixing is performed by transferring the dry thrombin of a) and the paste of b) back and forth between the connected containers thereby obtaining a substantially homogenous distribution of thrombin.

3. The method according to claim 1, wherein the number of transfers is less than 10 time.

4. The method according to claim 1, wherein the homogenous distribution of thrombin is characterized by a variation in thrombin content throughout the haemostatic composition of less than 20%.

5. The method according to claim 1, wherein the haemostatic composition is a paste suitable for use in haemostasis and/or wound healing.

6. The method according to claim 1, wherein the haemostatic composition is a flowable composition.

7. The method according to claim 1, wherein the dry thrombin composition is prepared by spray-drying or freeze-drying.

8. (canceled)

9. (canceled)

10. The method according to claim 1, wherein the biocompatible polymer consists of powder particles which are substantially insoluble in an aqueous medium.

11. The method according to claim 1, wherein the biocompatible polymer is cross-linked.

12. The method according to claim 1, wherein the biocompatible polymer is biologically absorbable.

13. The method according to claim 1, wherein the biocompatible polymer is gelatine, collagen, chitin, chitosan, alginate, cellulose, oxidised cellulose, polyglycolic acid, or polyacetic acid, or combinations thereof.

14. (canceled)

15. (canceled)

16. The method according to claim 1, wherein the paste in b) comprises the biocompatible polymer in a content of 10% w/w to 20% w/w.

17. The method according to claim 1 wherein the haemostatic composition comprises one or more hydrophilic compound(s).

18. The method according to claim 17, wherein the one or more hydrophilic compound(s) is one or more polyol(s).

19. (canceled)

20. (canceled)

21. (canceled)

22. (canceled)

23. (canceled)

24. The method according to claim 1, wherein the haemostatic composition comprises one or more further bioactive agents capable of stimulating haemostasis, wound healing, bone healing, tissue healing and/or tendon healing.

25. (canceled)

26. (canceled)

27. (canceled)

28. (canceled)

29. The method according to claim 1, wherein the first and the second containers are medical delivery devices suitable for reconstituting the dry thrombin composition and dispensing the haemostatic composition comprising thrombin.

30. The method according to claim 1, wherein the first and the second containers are syringes.

31. The method according to claim 1, wherein the first container is a glass syringe or wherein the first container comprises a glass insert comprising the dry thrombin composition.

32. (canceled)

33. The method according to claim 1, wherein the method comprises a further step of sterilising the dry thrombin composition in a), the paste in b) and/or the haemostatic composition.

34. (canceled)

35. (canceled)

36. A kit comprising
a) a first syringe comprising a dry thrombin composition;
b) a second syringe comprising a paste comprising a biocompatible polymer; and
c) optionally an outer package,
wherein the two syringes are interconnectable.

37. (canceled)
